(19) United States
(12) Patent Application Publication
(10) Pub. No.: US 2020/0038042 A1
(43) Pub. Date: Feb. 6, 2020

(54) TUMOR ENUCLEATOR AND METHOD OF USE

(71) Applicant: Yousef Sadeq Al-Shraideh, Duncansville, PA (US)

(72) Inventor: Yousef Sadeq Al-Shraideh, Duncansville, PA (US)

(21) Appl. No.: 16/528,064

(22) Filed: Jul. 31, 2019

Related U.S. Application Data
(60) Provisional application No. 62/712,644, filed on Jul. 31, 2018.

(51) Int. Cl. A61B 17/221; A61B 18/24

(52) U.S. Cl. CPC A61B 17/221; A61B 2017/320733; A61B 2017/2215; A61B 18/24

(57) ABSTRACT

An enucleator and method of use for resection of tumors, especially tumors of the bladder. The enucleator has a plurality of tentacles that are selective extensible and retractable from an elongate body. In a deployed, extended position, the tentacles may be positioned to surround the tumor and tumor stalk. The ends of the tentacles have a razor tipped edge that are oriented inwardly to surround. Utilizing the energy source at the tip of the razor ends, cut through the tumor stalk will finalize the enucleation en-bloc of the whole tumor in one piece.

### CROSS-REFERENCE TO RELATED APPLICATION

This application claims the benefit of priority of U.S. provisional application No. 62/712,644, filed Jul. 31, 2018, the contents of which are herein incorporated by reference

### BACKGROUND OF THE INVENTION

The present invention relates to urological surgical devices and techniques, and more particularly to bladder tumor enucleators and resection techniques.

Bladder cancer is the most common malignant disease involving the urinary system, which includes; the urothelium tissue from the renal pelvicalyceal system down to the level of the prostatic urethra (1). It comes after prostate cancer in incidence for the genitourinary system and comprises the fourth most common cancer in men and ninth most common cancer in women (2).

Bladder cancer accounts for around 400,000 new cancer cases, and 150,000 deaths annually. The rates vary, where the highest occur in Western Europe and North America, while the lowest rates found in Eastern Europe and Asia (3).

Older men are primarily affected with most cases diagnosed above the age of 65 years. Male to female ratio is 3:1, which may be higher in whites (4). The incidence in United States increased over 50% since 1985 (5).

Around 70 to 80% of the new urothelial bladder cancers are non-muscle invasive (NMIBC), which comprises the papillary form (Ta), subepithelial invasive (T1) and carcinoma in site (CIS). Most of the NMIBC are Ta which form around 70%, while T1 and Tis account for 20, and 10 percent, respectively (6).

The main stay of treatment of NMIBC is transurethral resection of the bladder tumor (TURBT), with adjuvant single intravesical instillation of chemotherapy in selected cases to decrease the risk of recurrence (7). Most tumors are papillary and are easily removed by the traditional electro-cutting loop in a piecemeal fashion. Medium and large tumors are resected piecemeal prior to transection of the stalk. Monopolar Transurethral Resection (TUR) was first invented by Stern in 1926, when he developed the resectoscope with electric cutting loop. Bipolar TUR then was invented early 2000s by Karl Storlz medical, which changed the electrical current from mono polar to bipolar, however, no change has happened to the apparatus itself or the procedure of tumor resection. Thus, since 1926, no major change has happened to the working apparatus of the TUR system and no alternative methods of resection were created.

Complications of the current TUR systems have affected the patient recovery and increased hospital stay, such as; bleeding, bladder perforation, TUR syndrome, and obturator reflex; specially with mono polar TUR, in addition to cautery artifacts which complicate pathological interpretation.

As can be seen, there is a need for an improved apparatus and methods for the treatment of bladder cancer, which should be more efficient and less side effects than the conventional TUR apparatus.

### SUMMARY OF THE INVENTION

In one aspect of the present invention, a tumor enucleator is disclosed. The tumor enucleator includes a handle having an elongate probe extending from an end thereof. An enucleator head is attached to a distal end of the probe. The enucleator head has a generally cylindrical body. A plurality of tentacles are radially disposed within the enucleator head. The plurality of tentacles are operable between a retracted position within the enucleator head and an extended position to protrude from the enucleator head.

In some embodiments, a plurality of controls are carried by the handle. Manipulation of the plurality of controls orients and guides the enucleator head.

A tip is provided at a distal end of each of the plurality of tentacles. In some embodiments, the tip is a sharpened cutting tip. In some embodiments, an energy source is provided and a conductor carries the energy source to the tip. The energy source may include a laser energy and the conductor may include a plurality of fiber optic cables. Preferably, the plurality of fiber optic cables are disposed transversely across the tip.

In some embodiments, an arcuate curvature is provided in each of the plurality of tentacles. In these embodiments, an encapsulating is basket is defined by the arcuate curvature of the plurality of tentacles when positioned in the extended position. In yet other embodiments, each tip is oriented for a resection at a base of a tumor stalk when the plurality of tentacles are retracted from the extended position to the retracted position.

Other aspects of the present invention include a method of resecting a tumor from within a body cavity. The method include providing a tumor enucleator having a handle with an elongate probe extending from a distal end thereof. An enucleator head is attached to a distal end of the probe, the enucleator head having a generally cylindrical body. A plurality of tentacles are radially disposed within the enucleator, head. The plurality of tentacles are operable between a retracted position within the enucleator head and an extended position.

The method also includes the step of inserting the enucleator head within the body cavity with the plurality of tentacles positioned in the retracted position. A control on the handle is manipulated to position the plurality of tentacles to the extended position. The handle is further manipulated to position the plurality of tentacles around a tumor growing within the body cavity.

Once positioned, the plurality of tentacles are retracted about the tumor. The tumor is then resected from the body cavity. The tumor enucleator is then manipulated to withdraw the enucleator head and the resected tumor from the body cavity.

In some embodiments, the method further includes communicating a laser light source to a tip of the plurality of tentacles via a fiber optic conductor carried within the plurality of tentacles. The tumor may then be resected with the laser light source.

In other embodiments, the method includes communicating an electrical charge to a tip of the plurality of tentacles. The tumor may then be resected with the electrical charge.

These and other features, aspects and advantages of the present invention will become better understood with reference to the following drawings, description and claims.

### BRIEF DESCRIPTION OF THE DRAWINGS

FIG. 1 is a front perspective view of a head of a tumor enucleator shown in a retracted position.

FIG. 2 is a front perspective view of the head of the tumor enucleator shown in an extended position surrounding a tumor.

FIG. 3A is a detail front perspective view of the tumor enucleator shown in the retracted position.

FIG. 3B is a detail front perspective view of the tumor enucleator shown in the extended position surrounding a tumor.

FIG. 4 is a side elevation view of the tumor enucleator and a perspective view of the tumor enucleator head.

FIG. 4A is a perspective view of the tumor enucleator head shown in the retracted position.

FIG. 5 is a side perspective view of a procedure inserting the tumor enucleator head into the bladder to resect a tumor.

FIG. 6 is a side perspective view of a procedure for resecting a bladder tumor.

FIG. 7 is a side perspective view of the tumor enucleator head in an expanded position to capture a tumor.

FIG. 8 is a side perspective view of the tumor enucleator head in an expanded position capturing a tumor.

FIG. 9 is a side perspective view of the tumor enucleator head withdrawing a tumor from the bladder.

FIG. 10 is a side perspective view of the tumor enucleator head inserted into the bladder.

FIG. 11 is a cross sectional view of a tentacle.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, embodiments of the present invention provide an improved apparatus and method for the treatment of bladder cancer.

The new and innovative bladder tumor enucleator 10, may be formed as a 30 cm long and 15-20 french (F) diameter probe 13 extending from a handle 11 having controls 15 to manipulate a head 12 of the enucleator 10. In use, the head 12 is inserted through the regular 24-28 F resectoscope sheath (not shown in the drawings for simplicity of illustration).

The head 12 of the enucleator 10 has a generally cylindrical body 14 which carries a plurality of 4-6 tentacles 16 radially disposed about a longitudinal axis of the enucleator head 12. The plurality of tentacles 16 terminate at a distal end with a flat razor tip 20. The plurality of tentacles 16 may also have an arcuate curvature or a pronounced bend at the distal end to define an encapsulating basket 22 and to orient the razor tip ends 20 for resection at the base of the tumor stalk T, creating the enucleation mechanism which is a core purpose of this invention.

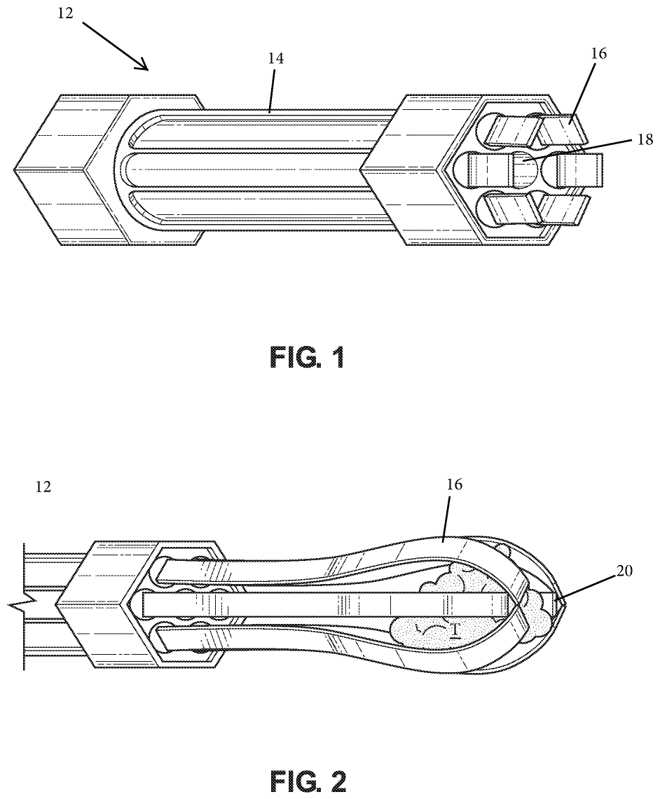

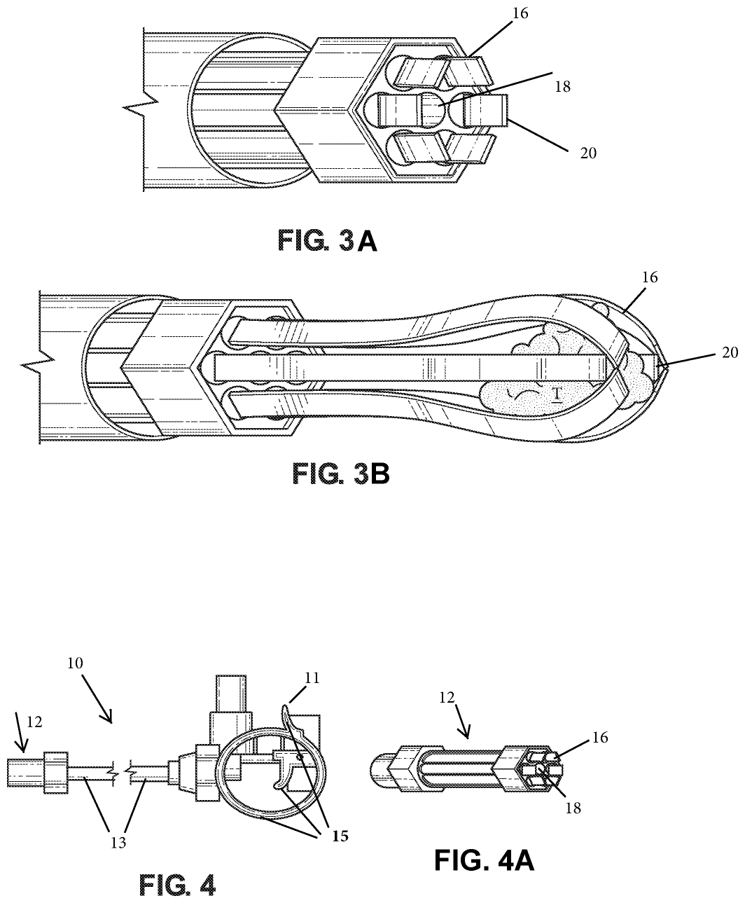

The plurality of tentacles 16 are operable between a back loaded, or retracted position, shown in reference to FIG. 1 and an extended, or deployed position, shown in reference to FIG. 2. When back loaded the tentacles 16 are carried in a body 14 of the main probe 10 during a non-functioning status. When extended and deployed, as shown in reference to FIG. 2, the tentacles 16 form a splayed diamond, or Jellyfish appearance, roughly around 3 cm in diameter. The splayed tentacles 16 are positioned to surround the target tumor T and the razor tips 20 are positioned to resect the tumor T at the tumor stalk.

Illumination and optics, such as a light and a camera 18, may be provided through a central axis of the enucleator 10 to provide visibility and illumination as the surgeon positions the plurality of tentacles 16 around the tumor. Alternatively, vision may also be provided through the regular 30-degree lens that runs through the conventional resectoscope sheath.

In some embodiments, an energy source is provided to facilitate cutting through the stalk of the tumor T and to control bleeding. The energy source could be an electrical energy or a laser energy. In the case of an electrical energy source, the electrical power can be communicated via the shaft 13 to the tentacles 16 and cutting tips 20.

In the case of a laser energy source, a laser light source may be communicated via a plurality of fiber optic cables 22 having a terminal end located at the cutting tips 20, as shown in reference to FIG. 11. In these embodiments, a holmium laser would be safer and more efficient, as the depth of energy penetration is very low (<0.5 mm). The Holmium laser setting starts from 1.5 Joule and frequency of 10 Hertz to give a final power of 15 Watt, which could be escalated to 20 Watt. Accordingly, the enucleator 10 should work best with a combination of mechanical cutting with the razor tip 20 and the laser or electrical energy directed via the fiber optic cables 22 in order to cut through the stalk of the tumor T and to achieve hemostasis (bleeding control), since the tumors T, which are robustly attached to the urinary bladder layers B, are very vascular and easily bleed.

The YouNed innovation functions mainly at the tumor T base (Stalk), so no piecemeal cuts are needed, as is the case with the conventional TUR loop devices and techniques. Thus, the risk of bleeding would be much less as the tumor T is resected in an en-bloc enucleation technique. The ability of the surgeon to resect the tumor T at the stalk would also lower the procedure time significantly and prevent the risk of post-operative bleeding and clot retention.

Utilizing the advantages of the apparatus and method of the present invention would eventually reduce the need or duration of a hospital stay, as the procedure will permit a one-day case. Similarly, the apparatus and method of the present invention, there will be a reduced reliance on long term need for a urinary catheter after the procedure. Consequently, it will reduce costs and efforts on patients and surgeons, respectively.

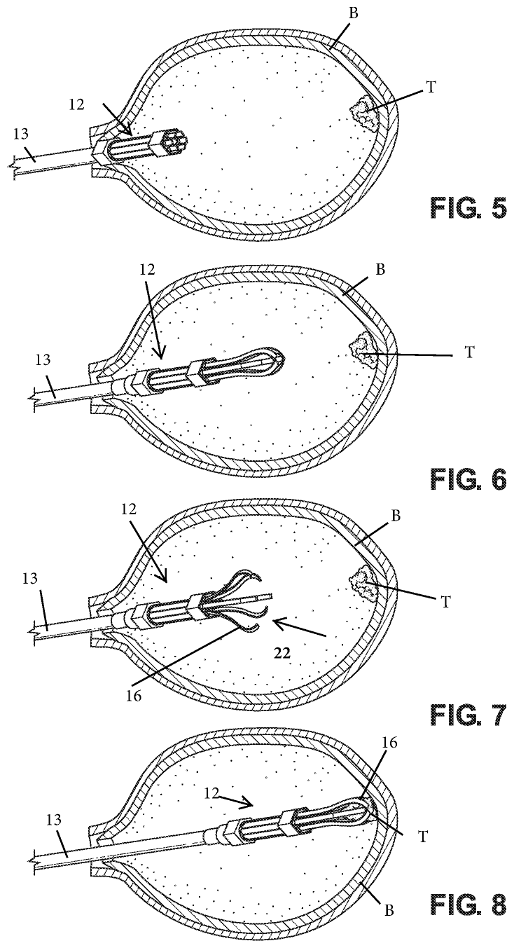

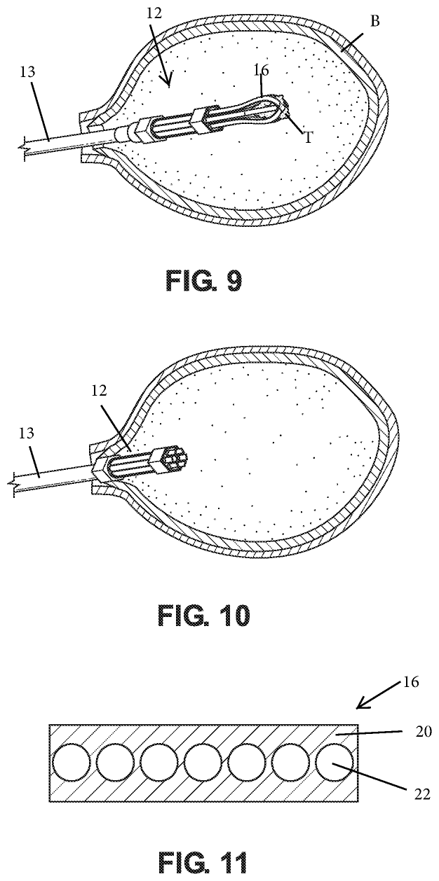

As shown in the simplified drawings of FIGS. 5-10, a urinary bladder tumor resection procedure is shown. During the operation of the YouNed enucleator 10, the bladder B should be filled with a fluid, but not over distended. After insertion of the enucleator head 12, the control 15 is manipulated to direct the head 12 towards the tumor T. The control 15 also permits the tentacles 16 to be splayed into a basket 22. With the tentacles 16 splayed, the handle 11 and controls 15 are manipulated to position the tentacles 16 at the correct angulation at the base of the tumor T, so the risk of bladder perforation would be reduced. The risk of bladder perforation is also reduced where the energy source would be the Holmium Laser, which has very low depth of penetration and eliminates the obturator reflex significantly as well.

After complete enucleation of the tumor T, deep tumor bed biopsies could be taken using cup biopsy forceps, to ensure a muscle layer of the bladder was appropriately sampled. This eliminates the cautery artifacts occur during the regular cutting TUR loop.

It should be understood, of course, that the foregoing relates to exemplary embodiments of the invention and that modifications may be made without departing from the spirit and scope of the invention as set forth herein and in the following claims.

### BIBLIOGRAPHY

1—Antoni S, Ferlay J, Soerjomataram I, et al. Bladder Cancer Incidence and Mortality: A Global Overview and Recent Trends. Eur Urol 2017; 71:96.
2—Kirkali Z, Chan T, Manoharan M, et al. Bladder cancer: epidemiology, staging and grading, and diagnosis. Urology 2005; 66:4.
3—Ploeg M, Aben K K, Kiemeney L A. The present and future burden of urinary bladder cancer in the world. World J Urol 2009; 27:289.
4—Torre L A, Bray F, Siegel R L, et al. Global cancer statistics, 2012. CA Cancer J Clin 2015; 65:87.
5—Siegel R L, Miller K D, Jemal A. Cancer statistics, 2018. CA Cancer J Clin 2018; 68:7.
6—Histological typing of urinary tumors, Mostofi F K, Sobin L H, Torloni H (Eds), World Health Organization, Geneva 1973.
7—Chang S S, Bochner B H, Chou R, et al. Treatment of Non-Metastatic Muscle-Invasive Bladder Cancer: AUA/ASCO/ASTRO/SUO Guideline. J Urol 2017; 198:552.

What is claimed is:

1. A tumor enucleator, comprising:
a handle having an elongate probe extending from an end thereof;
an enucleator head attached to a distal end of the probe, the enucleator head having a generally cylindrical body; and
a plurality of tentacles radially disposed within the enucleator, the plurality of tentacles operable between a retracted position within the enucleator head and an extended position.

2. The tumor enucleator of claim 1, further comprising:
a plurality of controls carried by the handle, wherein manipulation of the controls orient the enucleator head.

3. The tumor enucleator of claim 1, further comprising:
a tip at a distal end of each of the plurality of tentacles.

4. The tumor enucleator of claim 3, wherein the tip is a sharpened cutting tip.

5. The tumor enucleator of claim 3, further comprising:
an energy source and a conductor to carry the energy source to the tip.

6. The tumor enucleator of claim 5, wherein the energy source comprises a laser energy and the conductor comprises a plurality of fiber optic cables.

7. The tumor enucleator of claim 6, wherein the plurality of fiber optic cables are disposed across the tip.

8. The tumor enucleator of claim 1, further comprising:
an arcuate curvature in each of the plurality of tentacles.

9. The tumor enucleator of claim 8, further comprising:
an encapsulating basket defined by the arcuate curvature of the plurality of tentacles when in the extended position.

10. The tumor enucleator of claim 9, wherein each tip is oriented for a resection at a base of a tumor stalk when the plurality of tentacles retracted from the extended position to the retracted position. T, creating the enucleation mechanism which is a core purpose of this invention.

11. A method of resecting a tumor from within a body cavity, comprising:
providing a tumor enucleator comprising:
a handle having an elongate probe extending from an end thereof;
an enucleator head attached to a distal end of the probe, the enucleator head having a generally cylindrical body; and
a plurality of tentacles radially disposed within the enucleator, the plurality of tentacles operable between a retracted position within the enucleator head and an extended position.

12. The method of claim 11, further comprising:
inserting the enucleator head within the body cavity with the plurality of tentacles positioned in the retracted position.

13. The method of claim 12, further comprising:
manipulating a control on the handle to position the plurality of tentacles to the extended position.

14. The method of claim 13, further comprising:
manipulating the handle to position the plurality of tentacles around a tumor growing within the body cavity.

15. The method of claim 14, further comprising:
retracting the plurality of tentacles about the tumor.

16. The method of claim 15, further comprising:
resecting the tumor from the body cavity.

17. The method of claim 16, further comprising:
withdrawing the enucleator head and the tumor from the body cavity.

18. The method of claim 16, further comprising:
communicating a laser light source to a tip of the plurality of tentacles via a fiber optic conductor carried within the plurality of tentacles; and
resecting the tumor with the laser light source.

19. The method of claim 16, further comprising:
communicating an electrical charge to a tip of the plurality of tentacles; and
resecting the tumor with the electrical charge.
